(19) United States
(12) Patent Application Publication
(10) Pub. No.: US 2024/0382452 A1
(43) Pub. Date: Nov. 21, 2024

(54) COMPOSITIONS AND DOSAGE FORMS FOR TOPICAL AND TRANSDERMAL DELIVERY OF ACTIVES AND METHODS OF USE

(71) Applicant: OPHIREX, INC., Corte Madera, CA (US)

(72) Inventor: Matthew R. Lewin, Corte Madera, CA (US)

(21) Appl. No.: 18/703,760

(22) PCT Filed: Nov. 8, 2022
(86) PCT No.: PCT/US2022/049321

§ 371 Date: Apr. 23, 2024

Related U.S. Application Data
(60) Provisional application No. 63/277,252, filed on Nov. 9, 2021.

(51) Int. Cl. A61K 31/404; A61K 9/00; A61K 9/70; A61K 31/16; A61K 31/167; A61K 31/192; A61K 31/381; A61K 31/403; A61K 31/541; A61K 39/395; A61K 45/06; A61P 39/02

(52) U.S. Cl. CPC A61K 31/404; A61K 9/0014; A61K 9/7023; A61K 31/16; A61K 31/167; A61K 31/192; A61K 31/381; A61K 31/403; A61K 31/541; A61K 39/3955; A61K 45/06; A61P 39/02

(57) ABSTRACT

The present invention is directed to compositions comprising PLA2 inhibitors and optionally a metalloprotease inhibitor and/or an antivenom, antibody, antibody fragment or antibody derivative for topical, transdermal, buccal or sublingual administration, formulated especially as salves, creams, ointments, liniments, gels, films or tablets for topical, buccal or sublingual administration and patches and microneedle patches (which may include a massage or pressure pump to accelerate delivery and a band to secure the patch while providing sufficient pressure to inhibit the spread of toxin from a wound or envenoming site or the addition of an absorption accelerant incorporated into the device or added externally) for transdermal administration. The compositions according to the present invention may also be formulated with additional active agents such as antibiotics, steroids, analgesics, local anesthetics, additional actives and mixtures thereof to provide treatment for infections, wounds, injuries, inflammatory conditions and toxidromes, including those caused by bacteria, viruses, envenoming (venom-induced), especially snake and arthropod bites, as well as vesicant exposure and other wounds which traumatically arise from various causes at macro- and microscopic scales.

### FIELD OF THE INVENTION

The present invention is directed to compositions comprising PLA2 inhibitors and/or metalloproteinase inhibitors for topical, transdermal, buccal or sublingual administration, formulated especially as salves, creams, ointments and/or gels for topical administration used alone or in combination with an occlusive dressing and patches and microneedle patches (which may include a massage or pressure pump to accelerate delivery of active from a reservoir and a band to secure the patch and/or to provide sufficient pressure to inhibit the spread of toxin from a wound or envenoming site) for transdermal administration. The compositions according to the present invention may also be formulated with additional active agents such as antivenom (which includes humanized fragmented or single domain antibodies), antibiotics, steroids, analgesics, local anesthetic and mixtures thereof to provide treatment for infections, wounds, injuries, inflammatory conditions and toxidromes, including those caused by bacteria, viruses, fungi, malnutrition, envenoming (venom-induced), especially snake and arthropod bites, as well as vesicant exposure and other wounds which traumatically arise from various causes at macro- and microscopic scales.

### RELATED APPLICATIONS

This application claims the benefit of priority of United States provisional application U.S. 63/277,252 filed 9 Nov. 2021, the entire contents of said application being incorporated by reference herein.

### BACKGROUND AND OVERVIEW OF THE INVENTION

Preservation and/or accelerated healing of the basement layer membrane, epithelium, and endothelium is foundational for health in nearly every organ and tissue system of the body. These layers form the basic structure in nearly all tissues and organs. Destruction of these layers or loss of function leads to significant integumentary compromise. Unexpected and unique protection is created by compositions and kits related to combinations of sPLA2 and metalloprotease inhibitors applied individually and in combinations with additional agents such as antibiotics, steroids and analgesics.

### BRIEF DESCRIPTION OF THE INVENTION

The present invention is directed to topical, transdermal, sublingual and buccal formulations for treating a number of disease states and/or conditions, for example, infection (e.g. including cutaneous anthrax), inflammatory and wound conditions, including those wounds caused by bacteria, viruses, envenoming (venom-induced), especially snake and arthropod bites, as well as vesicant exposure and other wounds which traumatically arise from various causes at macro- and microscopic scales. In embodiments, the compositions according to the present invention are used to treat envenomation, including envenomation by a poisonous animal, especially a snake or arthropod. Pursuant to the present invention it has been discovered, quite surprisingly and unexpectedly that topical, transdermal, sublingual and buccal administration of compositions according to the present invention are particularly effective for treating the above-described disease states and/or conditions, especially envenomation from a snake or arthropod.

In the present invention, compositions comprise at least one PLA2 inhibitor alone or optionally in combination with a metalloprotease inhibitor and/or an antivenom (especially for example arthropod or snake antivenom, especially viper snake antivenom) or an antibody, antibody fragment or antibody derivative (preferably humanized) thereof as active(s) which optionally are formulated in combination with one or more additional active agents such as serine proteinase inhibitors and/or other actives including one or more of an acetylcholinesterase inhibitor (AChEI or AChEI combination), a spreading factor inhibitor, an NMDA receptor antagonist, an L-aminooxidase inhibitor, a muscarinic acetyl choline receptor (mAChR) inhibitor or a hyaluronidase inhibitor, among others. In embodiments, the AChEI is neostigmine. In embodiments, the composition comprises one or more of a local anesthetic, analgesic, antibiotic and/or steroidal compound as described herein. In embodiments, a local anesthetic such as lidocaine, prilocaine, bupivacaine, pramoxine, benzocaine, phenol, benzocaine, dibucaine or a mixture thereof. In embodiments, the use of lidocaine, prilocaine, bupivacaine or a mixture thereof is preferred.

The compositions according to the invention further comprise an absorption enhancement agent in effective amounts to assist the actives to penetrate the skin or mucosal membranes and deliver active through the skin (in the case of topical and/or transdermal administration) or mucosal tissue (in the case of buccal or sublingual administration). In embodiments, the absorption enhancement agent is DMSO, an alcohol, glycol, vegetable fatty oil, or a derivative thereof or a mixture thereof. In embodiments, the alcohol, glycol, vegetable fatty oil or derivative thereof includes, for example, ethanol, propanol, isopropanol, butanol, isobutanol, 2-butanol, tertiary-butanol, ethylene glycol, diethylene glycol, diethylene glycol monoester or diester, glycol ethers (such as diethyleneglycol monomethyl ether, diethyleneglycol monoethylether/transcutol), triethylene glycol, PEG 200, PEG 400, PEG 600, PEG 800, PEG 1000, PEG 1500, PEG 2000, propylene glycol, dipropyleneglycol, tripropylene glycol, a propylene glycol monoester or diester, a polypropylene such as PPG 200, PPG 400, PPG 600, PPG 800, PPG 1000, PPG 1500, glycerin or an ester thereof (e.g., monoacetin, diacetin or triacetin, preferably triacetin), a vegetable oil such as castor oil, coconut oil, soybean oil, corn oil, olive oil, bees wax, or a mixture thereof. In embodiments, the alcohol, glycol, fatty oil or derivative thereof is ethanol, propylene glycol, polypropylene glycol (often PPG 200, 400 or 600), diethyleneglycol monoethylether, castor oil, coconut oil or a mixture thereof.

In embodiments, the absorption enhancement agent is selected from the group consisting of DMSO, ethanol, stearic acid, sodium dodecyl sulfate (SDS), sodium lauryl sulfate (SLS), azone (I-dodecylazacycloheptan-2-one), carboxypolymethylene, polyoxyethylene fatty acid ester (polyolfatty acid ester), propylene glycol, fatty acids, esters of ricinoleic acid (e.g., methyl, ethyl, isopropyl, glycol, glyceryl, cetyl and octyldodecyl esters), castor seed oil, coconut oil, beeswax, shea butter, soybean oil, sweet almond oil, jojoba butter, tocopherol, citral, citonelloi, coumarin, farnesol, geraniol, limonene, linalool, isopropyl alcohol, mineral oil, polyoxyl 20 cetostearyl ether, propylene glycol, purified water, ammonia and mixtures thereof. In embodiments, DMSO is used as the absorption enhancement agent. In embodiments, DMSO is the absorption enhancement agent which is used alone or often in combination with ethanol. In embodiments of the present invention, ethanol is both an absorption enhancement agent as well as an agent which can be used to raise the freezing point depression of DMSO which is favorable for formulation, storage stability and administration of actives pursuant to the present invention.

In embodiments, the absorption enhancement agent is included in compositions according to the present invention in an effective amount of 0.1 to 50% or more by weight, 0.5% to 50% or more by weight, often 0.5% to 25%, often 1.5% to 20%, 1.5% to 15%, 2.5% to 10%, 5.0% to 15%, 2.5% to 10%, often 2.5 to 7.5% or 2.5 to 5% by weight. In embodiments, the absorption enhancement agent is DMSO which is often combined with an additional absorption enhancement agent as described herein. In embodiments, a combination of DMSO with at least one skin absorption agent, often ethanol, is preferred.

In embodiments, the PLA2 inhibitor is varespladib, methyl varespladib, AZD2716- 3-(5′-benzyl-2′-carbamoyl-[1,1′-biphenyl]-3-yl)-2-methylpropanoic acid (as a racemic mixture containing both “R” and “S” enantiomers, or as the enantiomerically enriched “R” or “S” enantiomer, preferably the racemic mixture, ((9-[(phenyl)methyl]-5-carbamoylcarbazol-4-yl) oxyacetic acid (LY433771), 3-(5′-benzyl-2′-carbamoyl-[1,1′-biphenyl]-3-yl)-propanoic acid (compound 4 of Giordanetto, et al., ACSMed. Chem. Lett., 2016, 7, 884-889, a pharmaceutically acceptable salt thereof or a mixture thereof. In embodiments, the composition also includes an effective amount of a metalloprotease inhibitor and/or a low molecular weight fragmented antibody or single domain antibody (e.g. a humanized camelid fragmented antibody or single domain antibody) or an antivenom (especially a viper snake antivenom). In embodiments, the metalloprotease inhibitor is prinomastat, marimastat, batimastat, vorinostat, ilomastat, tanomastat or a pharmaceutically acceptable salt or mixture thereof. In embodiments, the metalloprotease inhibitor is prinomastat, marimastat, batimastat, vorinostat or a mixture thereof, often prinomastat, marimastat or batimastat. Often, the metalloprotease inhibitor is prinomostat or marimastat, alone or in combination with another metalloprotease inhibitor.

In embodiments, the compositions according to the present invention include a mixture of DMSO and at least one additional absorption enhancement agent, often at least ethanol at a weight ratio range of DMSO:additional absorption enhancement agent (including ethanol) of 25:75 to 99:1, 50:50 to 99:1, often 75:25 to 97.5:2.5, often 80:20 to 95:5, more often 85:15 to 92.5:7.5, often about 80:20 or about 75:25.

In embodiments, the absorption enhancement agent is often ethanol, propylene glycol, polypropylene glycol (often PPG 200, 400 or 600), diethyleneglycol monoethylether, an ester of ricinoleic acid, castor oil, coconut oil or a mixture thereof. In embodiments, the absorption enhancement agent is ethanol and optionally an additional absorption enhancement agent.

It has unexpectedly been discovered pursuant to the present invention that DMSO/ethanol as an absorption enhancement agent is a good solvent which produces a depressed freezing point of DMSO (which is advantageous for formulation and administration of compositions according to the present invention by solubilizing and maintaining in solution the various active agents and other components included in the present compositions). Quite unexpectedly, it has been found that DMSO itself has substantial activity as a metalloprotease inhibitor in transdermal and topical applications pursuant to the present invention. This produces enhanced efficacy compared to a PLA2 inhibitor and/or a metalloprotease inhibitor in the absence of DMSO. Accordingly, in embodiments wherein the composition is to be used for the treatment of envenomation (often a snake or arthropod envenomation), DMSO is included as an absorption enhancement agent, alone or preferably in combination with ethanol and optionally, an additional absorption enhancement agent as otherwise disclosed herein.

In embodiments, the composition also comprises an anesthetic such as lidocaine, prilocaine, bupivacaine, pramoxine, benzocaine, phenol, benzocaine, dibucaine or a mixture thereof. In embodiments, the composition comprises DMSO and ethanol and also includes an anesthetic such as lidocaine, prilocaine, bupivacaine, pramoxine, benzocaine, phenol, benzocaine, dibucaine or a mixture thereof, often lidocaine, prilocaine, bupivacaine or a mixture thereof in amounts ranging from 0.1% to 20%, 0.5% to 15%, 1% to 12.5%, 1.5 to 10% by weight, often approximately 2.5% to 7.5% by weight, often about 5% by weight of the final composition.

In embodiments, the composition also includes an additional absorption enhancement agent as otherwise described herein in amounts ranging from 0.5% to 20%, 0.5% to 15%, 0.5% to 7.5%, often 1% to 5%, often approximately 2.5-3.0% by weight of the final composition. In embodiments, the composition comprises diethyleneglycol monoethylether, an ester of ricinoleic acid or a vegetable oil as an additional absorption enhancement agent.

In embodiments, active agents are used in effective amounts. Final compositions often range from about 0.025% percent to 75% by weight or more, 0.05% to 35% by weight or more, 0.075% to 20% by weight, 0.1% to 15% by weight, often 0.15% to 10% by weight or 0.25 to 7.5% by weight, more often 0.25% to 5%, 0.5% to 3.5-4.0% or about 2.5% by weight of active(s), with the remainder of the composition comprising an absorption enhancement agent as described herein, often, DMSO and an alcohol (often ethanol or isopropanol, more often ethanol), glycol, ester of ricinoleic acid, vegetable oil, a derivative thereof or a mixture thereof and any other additional agent(s) (non-active(s)) which facilitates formulation of the final compositions.

In embodiments, additional agents (non-actives) for inclusion in compositions according to the present invention include, for example, one or more pharmaceutically acceptable surfactants (e.g. non-ionic, cationic and/or anionic surfactants, often non-ionic surfactants including the Tweens, such as Tween 80/sorbitan monooleate, Tween 21/sorbitan monolaurate and Tween 40/sorbitan monopalmitate, among others), emulsifying agents (agar, albumin, alginates, casein, ceatyl alcohol, cholic acid, desoxycholic acid, diacetyl tartaric acid esters, glycerol, gums, carrageenan, lecithin, mono- and diglycerides, monosodium phosphate, monostearate, propylene glycol, soaps and taurocholic acid or its sodium salt), stabilizers (e.g., lecithin, agar-agar, carrageenan and pectin, to maintain compositions including emulsions in stable form), bulking agents (e.g., collagen and/or colloidal silicone dioxide, among others including Carbomer 934P NF Polymer, Cetyl alcohol, Candelilla/jojoba/rice bran polyglyceryl-3-esters, Beheneth-5, Calcium aluminum borosilicate, Wheat germ glycerides, Enteromorpha compressa extract, Ethyl macadamiate, Ethylhexyl stearate, Euphorbia cerifera wax, Zinc stearate, Xanthan gum, Sylitol, Sorbeth-230 tetraoleate, Sorbitan oleate, Sorbitan sesquioleate, Sortiban stearate, Sorbitol, Spirulina, Stearates, Isohexadecane, Stearyl alcohol, Stearic acid, Isofaraffin, Isopropyl lanolate, Tragacanth, Isostearamide DEA, Isostearic acid, Tribehenin, Tridecyl trimellitate, Triglyceride, Trihydroxystearin, Trilaurin, Trioctanoin, Kelp extract, Tridecyl stearate, Laminaria digitata, Laminaria longicruris, Liminaria sacharrina, Laureth-23, Laureth-4, Laureth-7, Gelatin, Lithium magnesium sodium silicate, Locust bean, Glycereth-26, Glycereth-26 phosphate, Glycereth-6 laurate, Glycerol monostearate, Glycerol triacetate, Glycerol trioleate, Glycerl behanate, Gyceryl cocoate, Sclerotium gum, Seamollient, Seaweed, Glyceryl dipalmitate, Glyceryl distearate, Glyceryl ester, Glyceryl isopalmitate, Glyceryl isostearate, Sodium carboxymethyl beta-glucan, Himanthalia elongate extract, Sodium lauroyl lactylate, hydroxyethylcellulose, hydroxypropyl starch phosphate, Irish moss extract, Ethanol (in Xanthan gum), Carbomer homopolymer Type C, cocoyl caprylocaprate, ), gelling agents (e.g., acacia, alginic acid, bentonite, Carbopols®/carbomers, carboxymethyl cellulose, ethylcellulose, gelatin, hydroxyethyl cellulose, hydroxypropyl cellulose, magnesium aluminum silicate, methylcellulose, poloxamers, polyvinyl alcohol, sodium alginate, tragacanth, and xanthan gum, among others), thickeners (e.g., starches, pectins and gums, among others), foaming agents, emollients (e.g., plant oils, mineral oil, shea butter, cocoa butter, petrolatum, and fatty acids such as animal oils, including emu, mink, and lanolin), antioxidants (e.g., vitamin E, vitamin C, vitamin C esters, glutathione, ubiquinone, astaxanthin, among others), preservatives (e.g., benzoates, sorbates such as potassium sorbate, calcium sorbate and sodium sorbate, propionates, butylated hydroxyanisole/BHA, BHT, tocopherol, ascorbic acid, ascorbyl palmitate, disodium ethylene diamine tetraacetic acid/EDTA, polyphosphates and citric acid, among others) and solvents, especially water, an alcohol, glycerin, a glycerin ester (monoacetin, diacetin or triacetin, often triacetin) and propylene glycol, among others.

Compositions according to the present invention are formulated into solutions, ointments, salves, creams, gels, lotions, pastes, powders, tablets, sprays, films and liniments (often ointments, gels or lotions for topical or transdermal administration and powders, pastes, gels and tablets, sprays or films for buccal or sublingual administration) or are formulated for transdermal delivery in patches and dissolvable microneedles. Transdermal patches often include a massage pump or finger pump which can be used at the wound site to accelerate drug dispersal and inhibit the spread of toxin from the direct area of the wound. Microneedles include (i) solid microneedles for skin pretreatment to increase skin permeability, (ii) microneedles coated with drug that dissolves off in the skin, (iii) polymer microneedles that encapsulate drug and fully dissolve in the skin and (iv) hollow microneedles for drug infusion into the skin. Topical administration often includes an occlusive dressing or bandage to facilitate administration and absorption of actives from the composition.

In embodiments, the present invention is directed to compositions and methods which evidence that LY315920, LY333013 and related sPLA2 inhibitors as described herein are particularly effective. In embodiments, the PLA2 inhibitor is varespladib (LY315920), methyl varespladib (LY333013), AZD2716-3-(5′-benzyl-2′-carbamoyl-[1,1′-biphenyl]-3-yl)-2-methylpropanoic acid—as the racemic mixture or as the “R” or “S” enantiomer, often as the racemic mixture, LY433771 ((9-[(phenyl)methyl]-5-carbamoylcarbazol-4-yl) oxyacetic acid) or 3-(5′-benzyl-2′-carbamoyl-[1,1′-biphenyl]-3-yl)-propanoic acid (Compound 4 of Giordanetto, et al., ACSMed. Chem. Lett., 2016, 7, 884-889 which reference is incorporated in its entirety herein, alternatively referred to as Compound 4 herein), a pharmaceutically acceptable salt thereof or a mixture thereof. In embodiments, the metalloprotease inhibitor is prinomastat, batimastat, marimastat or vorinostat, among others dosed alone or in combination (prinomastat, marimastat and batimastat are often the metalloproteases of choice depending on the desired half-life of the drug or the region of distribution) with preferred sPLA2 inhibitors for the treatment of wounds as otherwise described herein. In embodiments, a low moleular weight fragmented or single domain antibody (e.g., a canelid fragmented or single domain antibody), or an antivenom (e.g., a snake antivenom, including a viper snake antivenom) is often included in the formulation. In those embodiments, the snake antivenom is often a snake antivenom.

In embodiments, the compositions pursuant to the present invention, in topical or transdermal dosage form may be used to treat infection (e.g. including cutaneous anthrax), inflammatory and wound conditions, including those wounds caused by bacteria, viruses, envenoming (venom-induced), especially snake and arthropod bites, as well as vesicant exposure and other wounds which traumatically arise from various causes at macro- and microscopic scales.

Additional embodiments include the addition of topical anesthetics such as lidocaine, prilocaine, bupivacaine, pramoxine, benzocaine, phenol, benzocaine, dibucaine, often lidocaine, prilocaine, bupivacaine or a mixture thereof in effective amounts ranging from 0.1% to 10% by weight of the final composition, 0.5% to 7.5% by weight, 0.5% to 5% by weight, often 1% to 5%, often 1.5% to 3.5% or about 2.5% by weight for topical anesthesia and lymphatic relaxation to slow toxin spread subsequent to envenomation and antibiotics or antivirals for infection, such as an anthrax or other bacterial or viral infection. An occlusive dressing or bandage may further slow toxin spread. Preferred antibiotics which find use in the present invention include ciprofloxacin, levofloxacin, moxifloxacin, penicillin G, doxycycline, macrolides and chloramphenicol, ofloxacin and mixtures thereof. In embodiments, an analgesic or topical steroid is optionally included in the composition. Representative analgesics include the salicylates, along with capsaicin, menthol, and camphor, among others. Representative topical steroids include amcinonide, betamethasone dipropionate, clobetasol propionate, desoximetasone, diflorasone diacetate, halobetasol propionate and triamcinolone, among several others.

In an embodiment, the present invention is directed to methods for treating infections and inflammatory and wound conditions, including wounds caused by bacteria, viruses, envenoming (venom-induced), especially snake and arthropod bites, as well as vesicant exposure and other wounds which traumatically arise from various causes at macro- and microscopic scales in a patient or subject in need comprising administering to the site of the infection and/or condition of the patient or subject an effective amount of a composition as otherwise described herein, either topically or transdermally. In certain embodiments, the compositions are administered at the site of the infection and/or inflammatory or wound condition on the skin of the patient or subject by application of the topical composition directly to the area or near the area of the infection and/or wound or alternatively, by transdermal patch at or near the site of infection and/or inflammatory and/or wound condition where there is a massage or pressure pump included with the patch to accelerate delivery and a band to secure the patch and/or provide sufficient pressure to inhibit the spread of toxins from a wound or envenoming site.

In embodiments, compositions often comprise varespladib sodium, AZD2716 as a racemic mixture referred to herein, compound 4 or a mixture thereof as a PLA2 inhibitor, often varespladib Na+, often in combination with a metalloprotease inhibitor such as prinomastat, marimistat or bastimistat often at a molar ratio ranging from 1:1 to 1:5, often about 1:2 used neat in DMSO or a combination of DMSO and ethanol (often 80:20) wherein the PLA2 inhibitor ranges from about 0.25 to 5% by weight of the composition, the metalloprotease inhibitor ranges from about 0.25 to 10% by weight of the composition with the remainder of the composition comprising DMSO alone or in combination with ethanol (often at 80:20 DMSO to ethanol). In embodiments, these compositions include lidocaine, bupivacaine or prilocaine at a weight ratio ranging from 0.25% to 5%, often 0.5% to 4.5% or 1% to 3% for analgesia and to slow lymphatic spread of toxins.

In embodiments, compositions comprise a PLA2 inhibitor (e.g., varespladib sodium, AZD2716 3-(5′-benzyl-2′-carbamoyl-[1,1′-biphenyl]-3-yl)-2-methylpropanoic acid—as the racemic mixture, compound 4 or a mixture thereof as a PLA2 inhibitor, often varespladib Na+) often in combination with a metalloprotease inhibitor such as prinomastat, marimistat or bastimistat often at a molar ratio ranging from 1:1 to 1:5, often about 1:2, wherein the PLA2 inhibitor ranges from about 0.25 to 5% by weight of the composition, the metalloprotease inhibitor ranges from about 0.25 to 10% by weight of the composition with the remainder of the composition comprising carboxypolymethylene as a thickening agent ranging from about 1% to about 20% by weight, about 60% to about 95% by weight of polyoxyethylene oxide fatty acid ester (ricinoleate/castor oil PEG such as PEG 60 hydrogenated castor oil, coconut oil PEG), optionally including water, DMSO and/or DMSO/ethanol (80:20) as the remainder of the composition. The final pH of the composition ranges from about 7.5 to about 10. In embodiments, these compositions include lidocaine, bupivacaine or prilocaine at a weight ratio ranging from 0.25% to 5%, often 0.5% to 4.5% or 1% to 3% for analgesia and to slow lymphatic spread of toxins.

In alternative embodiments, compositions comprise a PLA2 inhibitor (e.g., varespladib sodium, AZD2716 3-(5′-benzyl-2′-carbamoyl-[1,1′-biphenyl]-3-yl)-2-methylpropanoic acid—as the racemic mixture, compound 4 or a mixture thereof as a PLA2 inhibitor, often varespladib Na+) often in combination with a metalloprotease inhibitor such as prinomastat, marimistat or bastimistat often at a molar ratio ranging from 1:1 to 1:5, often about 1:2, wherein the PLA2 inhibitor ranges from about 0.25 to 5% by weight of the composition, the metalloprotease inhibitor ranges from about 0.25 to 10% by weight of the composition with the remainder of the composition comprising PEG 40 hydrogenated Castrol oil, as a thickening agent ranging from about 1% to about 20% by weight, about 60% to about 95% by weight of 3% cetyl alcohol, optionally including water, DMSO and/or DMSO/ethanol (80:20) as the remainder of the composition. The final pH of the composition is adjusted with sodium hydroxide and ranges from about 7.5 to about 10. In embodiments, these compositions include lidocaine, bupivacaine or prilocaine at a weight ratio ranging from 0.25% to 5%, often 0.5% to 4.5% or 1% to 3% for analgesia and to slow lymphatic spread of toxins.

In alternative embodiments, compositions comprise a PLA2 inhibitor (e.g., varespladib sodium, AZD2716 3-(5′-benzyl-2′-carbamoyl-[1,1′-biphenyl]-3-yl)-2-methylpropanoic acid as a racemic mixture, compound 4 or a mixture thereof as a PLA2 inhibitor, often varespladib Na+) often in combination with a metalloprotease inhibitor such as prinomastat, marimistat or bastimistat often at a molar ratio ranging from 1:1 to 1:5, often about 1:2, wherein the PLA2 inhibitor ranges from about 0.25 to 5% by weight of the composition, the metalloprotease inhibitor ranges from about 0.25 to 10% by weight of the composition with the remainder of the composition comprising polysorbate 20/emulsifying wax 80%, as a thickening agent ranging from about 1% to about 20% by weight often 5% to 20%, about 60% to about 95% by weight of 3% cetyl alcohol, optionally including water, DMSO and/or DMSO/ethanol (80:20) as the remainder of the composition. The final pH of the composition is adjusted with sodium hydroxide and ranges from about 7.5 to about 10. In embodiments, these compositions include lidocaine, bupivacaine or prilocaine at a weight ratio ranging from 0.25% to 5%, often 0.5% to 4.5% or 1% to 3% for analgesia and to slow lymphatic spread of toxins.

In alternative embodiments, compositions comprise a PLA2 inhibitor (e.g., varespladib sodium, AZD2716 3-(5′-benzyl-2′-carbamoyl-[1,1′-biphenyl]-3-yl)-2-methylpropanoic acid as a racemic mixture, compound 4 or a mixture thereof as a PLA2 inhibitor, often varespladib Na+) often in combination with a metalloprotease inhibitor such as prinomastat, marimistat or bastimistat often at a molar ratio ranging from 1:1 to 1:5, often about 1:2, wherein the PLA2 inhibitor ranges from about 0.25 to 5% by weight of the composition, the metalloprotease inhibitor ranges from about 0.25 to 10% by weight of the composition with the remainder of the composition comprising carbomer, as a thickening agent ranging from about 0.5% to about 20% by weight or 1% to about 3%, about 60% to about 95% by weight of hydrogenated castor oil, optionally including water. DMSO and/or DMSO/ethanol (80:20). The final pH of the composition is adjusted with sodium hydroxide and ranges from about 7.5 to about 10. In embodiments, these compositions include lidocaine, bupivacaine or prilocaine at a weight ratio ranging from 0.25% to 5%, often 0.5% to 4.5% or 1% to 3% for analgesia and to slow lymphatic spread of toxins.

When treating envenomation with compositions according to the present invention, sufficient material as a cream, lotion or liquid should be placed on and near the affected areas of the skin in order to inhibit or resolve the conditions and/or disease state to be treated. This is often between 10 grams to 60 grams of composition or more, depending upon the surface area of the skin to be treated.

The following features characterize embodiments of the present invention, among others:
The compositions according to the present invention provide mixtures for rapid absorption, toxin neutralization and tissue stabilizing interventions. DMSO-based and DMSO/ethanol based compositions are preferred.
The addition of a topical anesthetic reduces pain from the tissue insult and slows toxin spread.
Freezing point depression of DMSO with ethanol insures rapid and effective transcutaneous absorption through direct contact, microneedle patch or other device-skin contact devices mitigating concerns for DMSO solidification in ambient conditions. The inclusion of ethanol reduces the tendency of DMSO to solidify in certain formulations to enhance absorptivity of the actives.
DMSO is uniquely useful for its heretofore unrecognized, direct, anti-metalloprotease activity as well as its ability to carry large concentrations of drug substance through intact or compromised skin. This is completely unexpected. The combination of DMSO and ethanol maintains the ability of the compositions to have actives absorbed topically and/or transdermally, while depressing (lowering) the freezing point of the DMSO to promote ease of formulation.
No topical cream has been made that can actually save a life after envenomation. The above experiments evidenced real life rescue of animals on death's doorstep.
The effect of the compositions seems to be highly durable and out of proportion to pill/IV. Neurotoxic mice administered the present compositions showed no signs of relapse in some instances for more than 96 hours.
Topical formulations are superior to transdermal patch in ease of use and large surface area make this application as efficient as patch and substantially more cost effective. Topical applications often used except where required.
The topical formulations of the present invention reversed systemic signs/symptoms of envenoming even when applied remotely to the bite site.

These and/or additional aspects of the present invention may be readily gleaned from the detailed description of the invention which follows.

### DETAILED DESCRIPTION OF THE INVENTION

The following terms shall be used throughout the specification to describe the present invention. Where a term is not specifically defined herein, that term shall be understood to be used in a manner consistent with its use by those of ordinary skill in the art.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention. In instances where a substituent is a possibility in one or more Markush groups, it is understood that only those substituents which form stable bonds are to be used.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

It must be noted that as used herein and in the appended claims, the singular forms “a,” “and” and “the” include plural references unless the context clearly dictates otherwise.

Furthermore, the following terms shall have the definitions set out below.

The term “patient” or “subject” is used throughout the specification within context to describe an animal, generally a mammal, especially including a domesticated animal and preferably a human, to whom a treatment, including prophylactic treatment (prophylaxis) is provided. For treatment of those infections, conditions or disease states which are specific for a specific animal such as a human patient, the term patient refers to that specific animal. In most instances, the patient or subject is a human patient of either or both genders.

The term “compound” is used herein to describe any specific compound or bioactive agent disclosed herein, including any and all stereoisomers (including diasteromers), individual optical isomers (enantiomers) or racemic mixtures, pharmaceutically acceptable salts and prodrug forms. The term compound herein refers to stable compounds. Within its use in context, the term compound may refer to a single compound or a mixture of compounds as otherwise described herein.

The term “effective” is used herein, unless otherwise indicated, to describe an amount of a compound or component which, when used within the context of its use, produces or effects an intended result, whether that result relates to the prophylaxis and/or therapy of an infection and/or disease state or as otherwise described herein. The term effective subsumes all other effective amount or effective concentration terms (including the term “therapeutically effective”) which are otherwise described or used in the present application.

The term “pharmaceutically acceptable” as used herein means that the compound, composition, including a salt form, is suitable for administration to a subject to achieve the treatments described herein, without unduly deleterious side effects in light of the severity of the disease and necessity of the treatment.

The terms “treat”, “treating”, and “treatment”, etc., as used herein within context, also refers to any action providing a benefit to a patient at risk for any of the disease states or conditions (which can be treated pursuant to the present invention (e.g., ameliorate, inhibit, reduce the severity, cure, etc.). Treatment, as used herein, principally encompasses therapeutic treatment, but may also encompass both prophylactic and therapeutic treatment, depending on the context of the treatment. The term “prophylactic” when used in context, means to reduce the likelihood of an occurrence or in some cases, reduce the severity of an occurrence within the context of the treatment of a disease state or condition otherwise described herein.

The term “coadministration” shall mean that at least two compounds or compositions are administered to the patient at the same time, such that effective amounts or concentrations of each of the two or more compounds may be found in the patient at a given point in time. Although compounds according to the present invention may be co-administered to a patient at the same time, the term embraces both administration of two or more agents at the same time or at different times, provided that effective concentrations of all coadministered compounds or compositions are found in the subject at a given time. Compounds and compositions according to the present invention may be administered with one or more additional bioactive agents to address specific disease conditions in a treated patient or subject.

The term “prevention” is used within context to mean “reducing the likelihood” of a condition or disease state from occurring as a consequence of administration or concurrent administration of one or more compounds or compositions according to the present invention, alone or in combination with another agent. Thus, the term prevention is used within the context of a qualitative measure and it is understood that the use of a compound according to the present invention to reduce the likelihood of an occurrence of a condition or disease state as otherwise described herein will not be absolute, but will reflect the ability of the compound to reduce the likelihood of the occurrence within a population of patients or subjects in need of such prevention.

Certain abbreviations are used to describe the present invention. As used herein, “AChE” is an abbreviation for acetylcholine; “AChEI” is an abbreviation for acetylcholinesterase inhibitor; “mAChR” is an abbreviation for muscarinic acetylcholine receptor; “nAChR” is an abbreviation for nicotinic acetylcholine receptor; Inhibitors of AChE that may also inhibit butyryl cholinesterases (BChE), pseudocholinesterases and others is implied. “MP” is an abbreviation for metalloproteinase (e.g., mammalian matrix metalloproteinase, MMPs, and snake venom metalloprotease, SVMPs; “SP” is an abbreviation for serine proteases; “MPI is an abbreviation for metalloproteinase inhibitor. Venoms (e.g., snake venoms) are secreted, and in the context of venoms “PLA2” and “sPLA2” are used interchangeably; PLA2-I is an abbreviation for phospholipase inhibitor; and “SPI” is an abbreviation for serine protease inhibitor.

The term “snakebite” includes “dry” snakebites as well as bites that result in envenomation or bite by non-venomous or unidentified snake. There are a number of ways to determine that a subject is a victim of a snake's bite. These include: the subject or another person witnessed the bite; physical evidence of snakebite (e.g., puncture wounds or lacerations, localized pain, local redness or swelling) is observed; the subject exhibits signs or symptoms consistent with snakebite envenomation (e.g., pain, redness, bleeding, or other evidence of envenomation such as weakness or paralysis); the subject exhibits signs or symptoms consistent with neurotoxic envenomation and has not been previously diagnosed with a condition other than neurotoxic envenomation that accounts for the signs or symptoms; venom or venom-activity has been detected (e.g., at the bite site, in urine or blood, using a snakebite venom/venom activity detection kit) or by assaying for elevated PLA2 activity. Visual identification of the snake may indicate that the subject has been bitten by a neurotoxic venomous snake even in the absence of fang or tooth marks (e.g. as with Bungarus bites).

The terms “signs” and “symptoms” of neurotoxic envenomation include paresthesia, drowsiness, dysconjugate gaze, small muscle paralysis which may result in ptosis (lid lag), weakness of neck muscles, dysphagia, mydriasis, fasiciculation, increased salivation, increased sweating, loss of muscle coordination, abdominal pain, difficulty speaking, nausea, difficulty swallowing and other bulbar palsies, and vomiting, hypotension, respiratory distress and respiratory muscle paralyses. In some cases the subject displays early signs of including early signs of neurotoxic envenomation, such as small muscle paralysis in the form of lid lag, dysconjugate gaze, difficulty swallowing and other bulbar palsies. Clinical assessments of muscle function of a subject who has suffered envenomation or who is suspected to have suffered envenomation include: visual acuity, ease of swallowing, ability to protrude the tongue, diction, and ability to raise the head completely off the bed for more than five seconds (neck flexion) and reduced peak respiratory flow

The term “venom” has its normal meaning and is a poisonous secretion of an animal, such as a snake, other reptile, amphibian, spider, scorpion, tick, cone snail, coelenterate (jelly fish), etc. transmitted by a bite or sting.

The term “antivenom”, “antivenin” or “antivenene” is a biological product used in the treatment of venomous bites or stings. Antivenom is generally created by milking venom from a venomous vertebrate such as a snake, lizard or fish or extracting it from an invertebrate such as a spider, tick, insect, coelenterate or mollusk. The venom is then diluted and injected into a horse, sheep, rabbit or goat or chicken eggs. The subject animal or egg will undergo an immune reaction to the venom, producing antibodies against the venom's antigenic molecules which can then be harvested from the animal's blood, refined and used to treat envenomation. Internationally, antivenoms must conform to the standards of the pharmacopoeia and the World Health Organization. “Paraspecific” antivenoms are those with actions or properties in addition to the specific one considered medically useful (e.g. to determine the paraspecific actions of an antivenom). In embodiments, paraspecific antivenoms, especially those prepared from viper venoms are often used in the present invention.

The term “envenomation,” refers to injection of venom into a victim as a result of a bite by a reptile, amphibian, arthropod, mollusk, cnidarian, insect, coelenterate or other venomous vertebrate or invertebrate animal and includes neurotoxic, non-neurotoxic envenomation, and envenomations of undetermined character, as well Cobra-spit ophthalmia. Examples of non-neurotoxic envenomation include hemotoxic, vasculotoxic, cardiotoxic, and myotoxic envenomation and are generically referred to in the descriptions below as “hemotoxic” or “cytotoxic” envenomation.

The term “neurotoxic envenomation,” refers to envenomation with a neurotoxic venom. Neurotoxic venoms include, for example and not limitation, venoms produced by venomous snakes.

The term “venomous snake” refers to a snake having venom with any proportion of neurotoxic, hemotoxic, vasculotoxic, myotoxic, cytotoxic and/or other toxic properties. For example and without limitation venomous snakes include Cobra, Krait, Russell's Viper, Mambas, Coastal Taipan, New Guinea Death Adder, Boomslang (a Colubrid), Rattlesnakes, Coral snakes, Sea snakes (Hydrophiinae) among many other. All vipers including rattlesnakes, Russell's viper, saw-scaled viper, lance-head vipers, European vipers and habu snakes and mamushi among many others are venomous. All told, there are approximately 600 venomous snake species identified with more than 200 being considered of medical and veterinary significance worldwide. It will be appreciated that venoms comprise complex mixtures of proteins and other substances with toxic properties. Thus venom of a neurotoxic venomous snake may comprise agents with hemotoxic, vasculotoxic, cardiotoxic, myotoxic and/or other toxic properties, including neurotoxins.

The term “pharmaceutically acceptable” as used herein means that the compound or composition is suitable for administration to a subject, including a human patient, to achieve the treatments described herein, without unduly deleterious side effects in light of the severity of the disease and necessity of the treatment.

The term “small molecule” as used herein, refers to a molecule with a molecular weight of less than about 2500, or less than about 1000, or less than about 750, or less than about 500.

In certain embodiments, the invention provides a method for treating or reducing the likelihood of neurotoxin-induced respiratory failure in a human subject (or other mammal) by determining that the subject is a victim of a snakebite or other envenomation and administering topically and/or transdermally a composition as otherwise disclosed herein which comprises an effective amount of a PLA₂ inhibitor(s), often varespladib, methylvarespladib, AZD2716-3-(5′-benzyl-2′-carbamoyl-[1,1′-biphenyl]-3-yl)-2-methylpropanoic acid—as a racemic mixture or as the “R” enantiomer of the racemate, preferably as the racemic mixture, LY433771 ((9-[(phenyl)methyl]-5-carbamoylcarbazol-4-yl) oxyacetic acid), compound 4, a pharmaceutically acceptable salt thereof or a mixture thereof, and other components as described herein.

Compositions according to the present invention may also include at least one “metalloproteinase inhibitor”. Useful metalloproteinase inhibitors include, but are not limited to, prinomastat, BB-94 (marimastat), BB-2516 (batimastat), vorinostat, ilomastat, tanomastat or a mixture thereof, which are often used. Prinomastat, marimastat or batimastat are often used and prinomastat is very often used in combination with compositions according to the present invention. Other metalloproteinase inhibitors that may be used in the invention include but are not limited to, cefixime, ginkgolide, TAPI-2, TAPI-1, EGTA, EDTA, Phosphoramidon, TAPI-0, Luteolin, alendronate, tanomastat, ilomastat, nafamostat, collagenase inhibitor 1, Ro-32-3555, lactobionic acid, o-phenantroline, ecotin, 4-epi-chlortetracycline, teracycline, doxycycline or related antibiotic with additional, salutary antimicrobial effect, n-dansyl-d-phenylalanine, 20[R]ginsenosideRh2, pro-leu-gly-hydroxymate, gm6001, actinonin, arp-100, MMP9 inhibitor I, MMP2 inhibitor I, SB-3CT, Thiorphan (DL), 4-epi-demeclocycline, zinc methacrylate, funalenone, naturally derived or synthetic short peptide-inhibitor of snake venom metalloproteinase and analogs, derivatives, pharmaceutically acceptable salts, enantiomers, diastereomers, solvates and polymorphs and mixtures thereof. It is noted that DMSO itself has unexpectedly been discovered to possess metalloproteinase inhibitor activity.

Compositions according to the present invention may also include a snake antivenom, or an antibody (e.g. monoclonal or polyclonal) or derivative thereof, often an antibody fragment or single chain antibody or derivative thereof (which is often humanized), often derived from a canelid. The following represents a non-limiting list of antivenoms for which a PLA2 inhibitor can improve outcomes by improving the efficacy of the antivenom formulations by prior administration, co-administration or co-formulation. It is an unexpected result that antivenom may also be formulated for topical administration in compositions according to the present invention and antivenom would exhibit the enhanced activity in combination with a PLA2 inhibitor, especially including varespladib, methyl varespladib, AZD2716 3-(5′-benzyl-2′-carbamoyl-[1,1′-biphenyl]-3-yl)-2-methylpropanoic acid preferably as the racemic mixture (compound 7), LY433771, compound 4, a pharmaceutically acceptable salt thereof or a mixture thereof. Representative snake antivenom include the following:
Pit Viper Antivenom, produced by Pfizer Inc, Pit Viper Antivenom (Crotalidae) is a Polyvalent equine antisera product which is a refined and concentrated preparation of serum globulins obtained by fractionating blood from healthy horses immunized with the following venoms: Crotalus adamanteus (Eastern diamond rattlesnake), C. atrox (Western diamond rattlesnake), C. durissus terrificus (tropical rattlesnake, Cascabel) and Bothrops atrox. Pit Viper Antivenom is indicated for the treatment of envenomation caused by bites of crotalids (pit vipers);
Anavip antivenom, Instituto Bioclon SA de CV, Rare Disease Therapeutics Inc: Anavip contains equine crotalinae F(ab′)2 immunoglobulin fragments as an active ingredient;
Anti-Snake Venom (ASV) by Indian Drugs & Pharmaceuticals Ltd. Anti-Snake Serum contains polyvalent equine snake venom immunoglobulins is indicated for the treatment of snakebites;
Anti-Vipers Serum VACSERA Snake Venom Poisoning by The Holding Company for Biological Products & Vaccines (VACSERA). Anti-Vipers Serum contains polyvalent viper venom antiserum. It is indicated for the treatment of viper bite.
Antivenom PFIZER INC. Antivenom (Micrurus fulvius) is a refined, concentrated, and lyophilized preparation of serum globulins obtained by fractionating blood from healthy horses that have been immunized with eastern coral snake (Micrurus fulvius) venom;
Antiviperino BIRMEX Snake Venom Poisoning, Laboratorios de Biologicos y Reactivos de Mexico S A de C V (Birmex). Antiviperino contains Vipera berus venom immunoglobulins;
ASVS-Africa Snake antivenom, Bharat Serums And Vaccines Limited, is a polyvalent snake antitoxin. It is a refined and concentrated preparation of serum globulins obtained by fractionating blood from healthy horses which are hyperimmunized by venoms of different snakes. ASVS-Africa is indicated for the treatment of envenomation caused by snake bites.

ASVS-Asia Snake antivenom, Bharat Serums And Vaccines Limited, Globulins, ASVS-Asia is a polyvalent snake venom antiserum. Snake venom antiserum is a refined and concentrated preparation of serum globulins obtained by fractionating blood from healthy horses which are hyperimmunized by venoms of different snakes;
Black Snake Antivenom CSL LTD, CSL Limited. Black Snake Antivenom is prepared from the plasma of horses immunized with the venom of the king brown snake (Pseudechis australis) also known as the mulga snake and then collecting that part of the horse's blood which neutralizes this poison. The venom of the king brown snake contains neurotoxins, myolytic toxins and an anticoagulant toxin. Black Snake Antivenom is used for the treatment of patients who exhibit manifestations of systemic envenoming following a bite by a king brown or mulga snake and related species and subspecies “paraspecific” antivenom use.

Bothrofav antivenom, Sanofi, Antibodies, Globulins, Immunoglobulins, Immunoproteins, Serum Globulins, BothroFav is an F(ab′)2-containing antiserum used for the treatment of envenomation of Bothrops lanceolatus snakes;
Brown Snake Antivenom, CSL Limited, Antibodies, Antitoxins, Biological Factors, Globulins, Immunoproteins, Peptides, Serum Globulins, Brown Snake Antivenom is prepared from the plasma of horses immunized with the venom of the brown snake (Pseudonaja textilis). Brown Snake Antivenom is indicated for the treatment of patients who exhibit manifestations of systemic envenoming following a bite by a snake of the genus Pseudonaja. This genus includes the Eastern brown snake, the dugite and the gwardar (Western brown snake);
Coralmyn, Instituto Bioclon SA de CV, Toxins, Biological, Coralmyn is a polyvalent anticoral fabotherapetic containing fragments F(ab′)2 and Fab of immunoglobulin G (IgG);
Serum Globulins, CroFab® (BTG) contains ovine crotalidae immunoglobulin fragments as an active ingredient. Crotalidae Polyvalent Immune Fab (Ovine) is a venom-specific Fab fragment of immunoglobulin G (IgG) that works by binding to and neutralizing venom toxins, facilitating their redistribution away from target tissues and their elimination from the body.
Death Adder Antivenom CSL LTD Snake Venom Poisoning, CSL Limited is prepared from the plasma of horses immunized with the venom of the death adder (Acanthophis antarcticus). Death Adder Antivenom is indicated for the treatment of patients who exhibit manifestations of systemic envenoming following a bite by a death adder;
Fav-Afrique, Sanofi, Globulins, FAV-Afrique is a polyvalent snake antivenom, elaborated by immunization of horses with venom from 10 different snake species among the most dangerous in Africa and belonging to Elapidae and Viperidae families. Only F(ab′)2 fragments are kept and purified. This serum is able to decrease the quantity of circulating venom and therefore its toxicity. Favirept (Sanofi Pasteur) is a polyvalent equine F(ab′)2 antivenom indicated for snakebite in the Middle East snakes: Bitis, Echis, Naja, Cerastes, and Macrovipera;
Freeze-dried Habu Antivenom produced by KAKETSUKEN. Freeze-dried Habu Antivenom contains Trimeresurus flavoviridis immunoglobulin of equine origin. Freeze-dried Mamushi Antivenom (KAKETSUKEN) is of equine origin;
Polyvalent Snake Antivenom CSL LTD polyvalent Snake Antivenom contains antibodies to the venom of the following snakes King brown snake (Pseudechis australis), Tiger snake (Notechis scutatus), Brown snake (Pseudonaja textilis), Death adder (Acanthophis antarcticus), Taipan (Oxyuranus scutellatus). The antivenom is prepared from the plasma of horses immunized with the venom of these snakes. It is indicated for the treatment of patients in Papua New Guinea and in all Australian states except Victoria and Tasmania;
Sea Snake Antivenom (CSL Limited). Sea Snake Antivenom is prepared from the plasma of horses immunized with the venom of the sea snake Enhydrina schistosa. Sea Snake Antivenom is indicated for the treatment of patients who exhibit manifestations of systemic envenoming following a bite by a sea snake;
Polyvalent Anti Snake Venom (ASV) by Serum Institute of India Ltd is a polyvalent anti-snake venom serum is prepared from hyperimmunized horses against the venoms of the four most commonly encountered venomous snakes in India;
BIOLOGICAL E by Biological E. Limited antivenom is a polyvalent preparation that is a pepsin-digested, refined and concentrated preparation obtained from equine hyper immune serum. It is used to neutralize the venom of Echis carinatus, Naja naja, Bangarus caeruleus and Vipera (Daboia) russelli.

VINS Bioproducts Ltd produces antiserum contains snake venom immunoglobulins (equine) as an active ingredient. Snake Venom Vaccine by United Company for Biological Industries Ltd (Bio Egypt is indicated as prophylactic vaccine against snake bites;
Taipan Antivenom by CSL LTD is a Taipan Antivenom prepared from the plasma of horses immunized with the venom of the taipan snake (Oxyuranus scutellatus) and similar preparations;
Tiger Snake Antivenom by CSL LTD is prepared from the plasma of horses immunized with the venom of the tiger snake (Notechis scutatus). Tiger Snake Antivenom is indicated for the treatment of patients who exhibit manifestations of systemic envenoming following a bite by a tiger, copperhead or black snake;
ViperaTAb contains Vipera berus venom immunoglobulins as an active ingredient. It is a polyvalent immune fab (Ovine), comprising of specific antibody fragments that bind and neutralize Vipera berus venom components, including proteases, lipases and cardiotoxins, facilitating their redistribution away from target tissues and subsequent elimination from the body;
Viperfav (Sanofi) is a polyvalent equine F(ab′)2 immunoglobulin antivenom indicated for the prevention of poisoning against European snakes (Vipera sp);
Snake Antiserum (KAMADA) Antivipmyn by Instituto Bioclon SA de CV contains snake venom immunoglobulin fragments (equine) as an active ingredient. It is a polyvalent anti snake fabotherapeutic containing fragments F(ab′)2 of immunoglobulin G (IgG) hyperimmunoglobulins.

Invertebrate antivenoms with efficacy being enhanced by PLA2 inhibitors used in compositions according to the present invention include:
Box Jellyfish Antivenom CSL LTD Jellyfish, CSL Limited Box Jellyfish Antivenom is prepared from the plasma of sheep immunized with the venom of the box jellyfish (Chironex fleckeri). Analatro (Instituto Bioclon SA de CV and Rare Disease Therapeutics Inc contains latrodectus venom F(ab′)2 immunoglobulin fragments as an active ingredient;
Aracmyn Plus by Instituto Bioclon SA de CV and Rare Disease Therapeutics Inc contains polyvalent spider venom F(ab′)2 immunoglobulin as active ingredient;
Funnel Web Spider Antivenom, CSL Limited: Funnel Web Spider Antivenom is a purified immunoglobulin (mainly immunoglobulin G), derived from rabbit plasma Atrax robustus;
Reclusmyn Instituto Bioclon SA de CV: Reclusmyn contains immune F(ab′)2 antivenom.

Red Back Spider Antivenom, CSL Limited. Red Back Spider Antivenom is prepared from the plasma of horses immunized with the venom of the female red back spider (Latrodectus hasselti);
Stonefish Antivenom (CSL Limited) is prepared from the plasma of horses immunized with the venom of the stonefish (Synanceia trachynis);
Scorgen is produced by Bharat Biotech International Limited. Scorgen consists of scorpion venom antiserum, which is of equine origin. This antiserum contains purified, enzyme refined and concentrated specific heterologous immunoglobulins;
Anascorp Instituto Bioclon SA de CV, Accredo Health Group Inc, Rare Disease Therapeutics Inc. Anascorp contains Centruroides immunoglobulin fragments (equine);
Anti-Scorpion Serum VACSERA Scorpion. Anti-Scorpion Serum is a purified polyvalent scorpion anti-serum;
Scorpifav Scorpion, Scorpifav is a polyvalent equine F(ab′)2 antivenom. Scorpifav is indicated for the prevention of poisoning against North African and Middle East scorpions (Androctonus, Leiurus, Buthus);
Scorpion venom antiserum Scorpion, VINS Bioproducts Ltd. Scorpion venom antiserum contains of scorpion venom immunoglobulin fragments (equine) as an active ingredient.
Scorpion venom antiserum is indicated for the treatment of scorpion bite; Scorpion Venom Vaccine Scorpion, United Company for Biological Industries Ltd (Bio Egypt). Scorpion Venom Vaccine is indicated as prophylactic vaccine against scorpion sting; Scorpion Venoms Antitoxin by United Company for Biological Industries Ltd (Bio Egypt).
Scorpion Venoms Antitoxin contains immunoglobulins against scorpion venom with the ability to neutralize toxins;
Stop Itch Skin Rash, Valeant Pharmaceuticals Australasia Pty Ltd. The active ingredient in Stop Itch is Papain, an enzyme extracted from the unripe fruit of paw paw (Carica papaya). Papain works two ways by inhibiting sensory transmission and neutralizing insect venom to quickly soothe and reduce the pain, itching and swelling associated with insect bites and minor skin irritations;
CSL Tick Antivenom Tick Antivenom helps to neutralize the effect of the Ixodes holocyclus venom. The antivenom is produced by immunizing dogs Tick Antivenom is indicated for the treatment of patients showing evidence of paralysis as a result of tick poisoning.

The term “antibody”, “antibody fragment” or “antibody derivative” is used to describe an antibody, antibody fragment, antigen-binding fragment (Fab), single chain variable fragment (scFV), single domain antibody or antibody derivative which is optionally included in compositions according to the present invention for their ability to bind to an epitope on venom, especially arthropod or snake venom and neutralize the venom which is introduced into a patient or subject from an envenomation. Exemplary antibodies, antibody fragments or antibody derivatives which may be included in compositions according to the invention include, for example, IgG, IgA, IgG, IgE, IgM, Fab, Fab2, scFv, dAb, Fc, Monomeric Fc, Hexameric Fc, Half antibody, scFv-Fc, dAb-Fc, Free LC, Long neck scFv-Fc, Minibody, Hexameric IgG, Fc fusion (N-term), Fc fusion (C-term), Monomer Fc fusion (N-term), Monomer Fc fusion (C-term), Hexameric Fc fusion, IgG-scFv (HC C-term), IgG-scFv (LC C-term), IgG-scFv (HC N-term), IgG-scFv (LC N-term), IgG-dAb (HC C-term), IgG-dAb (LC C-term), IgG-dAb (HC N-term), IgG-dAb (LC N-term), Protein IgG-fusion (HC C-term), Half antibody-scFv (HC C-term), Half antibody-scFv (LC C-term), scFv-Fc-scFv Heterodimeric IgG (common LC), Heterodimeric IgG (common HC), Heterodimeric IgG (HC+LC), Heterodimeric Fab/scFv-Fe, Heterodimeric Fab/dAb-Fc, Heterodimeric scFv-FcTandem, scFvFab-scFv (HC C-term), Fab-scFv (LC C-term), Fab-(scFv)2 (C-term), scFv-Fab-scFv (type 1), scFv-Fab-scFv (type 2), Fab-CH2-scFvIgG-scFv (HC+LC C-term), Heterodimeric IgG-scFv (type 1), Heterodimeric IgG-scFv (type 2), Heterodimeric IgG-scFv (type 3), TCBHeterodimeric Fab-scFv/scFv-Fc Heterodimeric IgG-fusion (type 1), Heterodimeric IgG-fusion (type 2), Non-antibody proteins, Heterodimeric IgG-fusion (type 3), scFv fusion (C-term), Heterodimeric Fc fusion (N-term), Heterodimeric Fc fusion (C-term), Bispecific Fc fusion (type 1), Bispecific Fc fusion (type 2), Bispecific Fc fusion (type 3), Heterodimeric bispecific Fc fusion (type 1), Heterodimeric bispecific Fc fusion (type 2), Heterodimeric bispecific Fc fusion (type 3), Heterodimeric bispecific Fc fusion (type 4), Heterodimeric Fab/protein Fc fusion, Trispecific Fc fusion (type 1), Tandem Fc fusion (type 1), Tandembody, Triple Fc fusion (type 1), Triplebody, Probody, Heterodimeric protein, Homodimeric protein, mAb280, Fcab81, Fab-fusion (type 1), Fab-fusion (type 2), Extended hinge IgG, IgA with J-chain, IgM with J-chain, Protein-protein fusion, Tandem Fc fusion (type 2) 1), Heterodimeric Fab-Fe, Peptide-IgG-scFv, Heterodimeric Fab-Fc-scFv/scFv-Fe, Heterodimeric Fab/protein Fc fusion (type 2), OctomAb, Heterodimeric VHH-Fe, Half antibody-scFv (HC+LC C-term), Heterodimeric IgG-scFv (type 4), scFv-scFv-dAb, scFv-dAb-scFv, dAb-scFv-scFv, scFv fusion (N-term), N-term Fc fusion-scFv, Trispecific Fc fusion (type 2), Tribody, Single chain homotrimer, Fab-IgG, Bispecific Fab-IgG, IgG-Fab, Minibody-scFv, Heterodimeric IgG-scFv (type 5), Tandem scFv-protein fusion, Fab-fusion (type 3), Fab-fusion (type 4), Protein-dAb-se homotrimer (type 1), Protein-dAb-se homotrimer (type 2), Protein-se homotrimer (type 1), Protein-se homotrimer (type 2), Heterodimeric BiTE-Fe, Trispecific dAb, Bispecific Fab2 (type 1), Bispecific Fab2 (type 2), Fab3, Fab4, Fab5, Fab6, Fab7, Heterodimeric IgG-scFv (type 6), IgY, Tetravalent monospecific IgG-scFv, Hexavalent monospecific IgG-scFv, scFv-monomeric Fe, Fc-scFv, Fab-VHH (HC C-term), Fab-VHH (LC C-term), Heterodimeric IgG-scFv (type 7), Fab8, Heterodimeric IgG-fusion (type 4), TandAb, Tandem Fab (domain swapped), DART, Heterodimeric protein-Fab/scFv-Fe, Heterodimeric IgG-fusion (type 5), Monospecific tandem scFv, Monospecific tandem scFv-fusion, IgG-dAb-protein fusion (type 1), Fc fusion protein (N and C-term), Tetravalent monospecific IgG-scFv (LC fusion), camelid.

Compositions according to the present invention optionally include a serine protease inhibitor. Useful serine protease inhibitors include, but are not limited to, nafamostat and gabexate, camostat or camostate. In certain instances cited herein, above and below, gabexate, camostat or camostate and other inhibitors directly or indirectly suppress snake venom or host PLA2 activity with salutary effect following snakebite or other envenomation. Other useful serine protease inhibitors include, but are not limited to, aprotinin, leupeptin, antithrombin (AT), alpha-1 antitrypsin (α₁-antitrypsin (A1AT)), AEBSF (4-(2-aminoethyl) benzenesulfonyl fluoride hydrochloride), PMSF (phenylmethanesulfonylfluoride or phenylmethylsulfonyl fluoride), protein C inhibitor (PCI, SERPINA5), protein Z-dependent protease inhibitor, methoxy arachidonyl fluorophosphonate (MAFP), myeloid and erythroid nuclear termination stage-specific protein (MENT), plasminogen activator inhibitor-1 (PAI-1), plasminogen activator inhibitor-2 (placental PAI), protease-nexin-1 (PN-1), antithrombin III colligin, phosphatidylethanolamine-binding protein, neuroserpin, α₂-antiplasmin, serine protease inhibitor 3, murinoglobin I, a naturally derived or synthetic short peptide-inhibitor of snake venom serine protease, ribozymes and small molecule agents that reduce the transcription or translation of a serine protease polynucleotide as described in U.S. Patent Application Document No. 20090318534, the serine protease inhibitors described or referenced in U.S. Patent Application Document No. 20140341881 and analogs, derivatives, pharmaceutically acceptable salts, enantiomers, diastereomers, solvates, polymorphs and mixtures thereof.

Compositions according to the present invention may further include an acetylcholinesterase inhibitor (AChEI) or a muscarinic acetylcholine receptor (mAChR) antagonist. Useful acetylcholinesterase inhibitors include, but are not limited to, ambenonium, demarcarium, donepezil, edrophonium, galantamine, huperzine A, ladostigil, lactucopicrin, neostigmine, physostigmine, pyridostigmine, rivastigmine, tacrine, phospholine iodide, ungeremine and mixtures thereof. Useful mAChR antagonists include competitive antagonists. The mAChR antagonist can be a reversible competitive antagonist and preferably does not cross the blood brain barrier. Preferably, the mAChR antagonist is selective for mAChR over nicotinic acetyhlcholine receptor (nAChR) and has a half-life of 4 to 6 hours or less. Useful mAChR agonists include, but are not limited to, glycopyrrolate and atropine, and preferred acetylcholinesterase inhibitor—mAChR antagonist co-therapy combinations include neostigmine and glycopyrrolate or atropine. Exemplary combinations of adjuvant therapeutics that can be co-administered with the PLA₂ inhibitor and optionally, an antivenom, a metalloprotease inhibitor and/or a serine protease inhibitor include phospholine iodide, an oxime-derived AChE restoring agent such as pralidoxime and a mAChR inhibitor such as atropine or biperiden. In one embodiment, the mAChR antagonist is selected from the group consisting of atropine, benzatropine, glycopyrrolate, ipratropium, mebeverine, oxybutynin, pirenzepine, scopolamine, biperiden, tiotropium and tropicamide.

Useful “NMDA receptor antagonists” which may be included in compositions according to the present invention include, but are not limited to, dizocilpine (MK801), ifenprodil, R025-6981, TCN-201, ketamine, fluorofelbamate, felbamate, memantine, dextromethorphan, eliprodil, selfotel, Conantokin-G, -R, aptigamel (CNS1102), dynorphin A(1-13), DQP 1105, and NVP-AAM077.

Useful antibiotics for inclusion in the present compositions include, but are not limited to, Demeclocycline, Doxycycline, Vibramycin Minocycline, Tigecycline, Oxytetracycline, Tetracycline, Gentamicin, Kanamycin, Neomycin, Netilmicin, Tobramycin, Paromomycin, Spectinomycin, Geldanamycin, Herbimycin, Rifaximin, Streptomycin, Ertapenem, Doripenem, Imipenem/Cilastatin, Meropenem, Cefadroxil, Cefazolin, Cephalothin, Cephalexin, Cefaclor, Cefamandole, Cefoxitin, Cefprozil, Cefuroxime, Cefixime, Cefdinir, Cefditoren, Cefoperazone Cefotaxime, Cefpodoxime, Ceftazadime, Ceftibuten, Ceftizoxime Ceftriaxone, Cefepime, Ceftaroline fosamil, Ceftobiprole, Teicoplanin, Vancomycin, Telavancin, Daptomycin, Oritavancin, WAP-8294A, Azithromycin, Clarithromycin, Dirithromycin, Erythromycin, Roxithromycin, Telithromycin, Spiramycin, Clindamycin, Lincomycin, Aztreonam, Furazolidone, Nitrofurantoin, Oxazolidonones, Linezolid, Posizolid, Radezolid, Torezolid, Amoxicillin, Ampicillin, Azlocillin, Carbenicillin, Cloxacillin Dicloxacillin, Flucloxacillin, Mezlocillin, Methicillin, Nafcillin, Oxacillin, Penicillin G, Penicillin V, Piperacillin, Temocillin, Ticarcillin, Amoxicillin/clavulanate, Ampicillin/sulbactam, Piperacillin/tazobactam, Ticarcillin/clavulanate, Bacitracin, Colistin, Polymyxin B, Ciprofloxacin, Enoxacin, Gatifloxacin, Gemifloxacin, Levofloxacin, Lomefloxacin, Moxifloxacin, Nalidixic acid, Norfloxacin, Ofloxacin, Trovafloxacin, Grepafloxacin, Sparfloxacin, Mafenide, Sulfacetamide, Sulfadiazine, Sulfadimethoxine, Sulfamethizole, Sulfamethoxazole, Sulfasalazine, Sulfisoxazole, Trimethoprim-Sulfamethoxazole, Sulfonamidochrysoidine, Clofazimine, Capreomycin, Cycloserine, Ethambutol, Rifampicin, Rifabutin, Rifapentine, Arsphenamine, Chloramphenicol, Fosfomycin, Fusidic acid, Metronidazole, Mupirocin, Platensimycin, Quinupristin/Dalfopristin, Thiamphenicol, Tigecycline and Tinidazole and combinations thereof.

In embodiments, particularly useful antibiotics treat snakebite-related microbes and act as venom neutralizing agents. Examples of such antibiotics include cephalosporins such as cefixime and tetracyclines such as doxycycline and demeclocycline.

In addition to the above components, compositions and methods according to the present invention may be enhanced by the inclusion of an effective amount of an agent which promotes biodistribution, such as, for example lidocaine, prilocaine or bupivacaine or other local anesthetic, such as pramoxine, benzocaine, phenol or dibucaine, often lidocaine, prilociane, bupivacaine or a mixture thereof which may be included in compositions according to the present invention to enhance distribution of the active components in the tissue into which the composition is injected while relaxing smooth muscle and slowing venom spread (as a spreading factor inhibitor).

Methods of treatment and pharmaceutical compositions of the invention can inhibit angiogenesis associated with snakebite through use of “spreading factor inhibitors”. Spreading factor inhibitors encompass inhibitors of the vitronectin receptor α₅α₃ including, but not limited to those vitronectin receptor α₅α₃ inhibitors described or referenced in U.S. Pat. No. 8,546,526, and those inhibitors of plasminogen activator inhibitor-1 (PAI-1; SERPINE1) described or referenced in Simone, et al., “Chemical Antagonists of Plasminogen Activator Inhibitor-1: Mechanisms of Action and Therapeutic Potential in Vascular Disease”, J Mol Genet Med, Volume 8, Issue 3, 1000125 (2014), which references are incorporated in their entirety herein. XR330, XR334, XR1853, XR5082, XR5967, XR1121, AR-H029953XX, fendosalanthranalic acid derivatives of the fibrinolytic antagonist flufenamic acid, ANS, bis-ANS, 1-dodecyl sulphurc acid, XR-5118 CDE-066, CDE-081, IMD-1622, Tiplaxtinin and TM5007 are useful spreading factor inhibitors. Useful spreading factor inhibitors also include naturally derived or synthetic short peptide-inhibitors of snake venom spreading factors and naturally derived or synthetic short peptide-inhibitor of non-enzymatic, tissue destructive, cardiotoxic, myotoxic, paralytic or hemorrhagic toxins. As discussed, lidocaine, prilocaine, bupivacaine, benzocaine and dibucaine and other topical anesthetic agents as described hereinabove can relax lymphatic smooth muscle and slow the spread of venom and can function as spreading factor inhibitors. Physical spreading factor inhibitors include pressure immobilization devices can be used in conjunction with the present invention to slow the spread of venom and increase the time window to definitive or adjunct treatment.

In an embodiment, the invention provides a method for treating or reducing the likelihood of tissue damage, bleeding and clotting disorders, cardiovascular collapse or neurotoxin-induced respiratory failure in a human or animal, comprising determining or clinically suspecting that the subject is a victim of a snakebite and administering pharmaceutically effective doses of inhibitors to the subject, wherein the inhibitor is administered by topical or transdermal administration.

Pursuant to the present invention, the absorption enhancement agent, often DMSO and ethanol and optionally an additional absorption enhancement, often an alcohol, glycol, vegetable oil and/or derivative thereof used in combination are admixed with actives (e.g., a PLA2 inhibitor and optionally, a metalloprotease inhibitor and/or a snake antivenom or antibody, antibody fragment or antibody derivative and optionally, one or more of a local anesthetic, analgesic, antibiotic and steroidal compound or other agent as described herein) to provide solubility of the individual components in the topical and/or transdermal formulations. DMSO has unexpectedly been discovered to be an excellent metalloprotease inhibitor itself and can be used in combination with a PLA2 inhibitor in the absence of an additional metalloprotease inhibitor or in combination with a metalloprotease inhibitor to enhance the effect, optionally in combination with other agents. The ratio of DMSO to skin absorption agent (e.g. alcohol, glycol or vegetable oil used, often DMSO and ethanol) is that amount effective to dissolve the actives for formulation and sufficiently depress the freezing point of DMSO so that the formulation may be maintained in a liquid, non-crystalline form for maximum delivery of active at the therapeutic site. Thus, depending on the skin absorption agent chosen to be used in combination with DMSO (often ethanol), the ratio of the DMSO to additional absorption enhancement agent (e.g. an alcohol, glycol, vegetable oil or derivative thereof most often ethanol, as described) will vary according to the weight ratio ranges which are described above.

The compositions comprising DMSO and skin absorption agent, often an alcohol, glycol, vegetable oil, a derivative thereof or a mixture thereof (most often ethanol) and active(s) are administered to a patient or subject in topical or transdermal dosage form. In the case of topical dosage forms, these compositions are formulated as salves, ointments, creams, liniments and/or gels using standard components known in the art. These compositions comprise a base composition which itself comprises DMSO and a skin absorption agent, often an alcohol, glycol, vegetable oil, a derivative thereof or mixtures thereof (often ethanol) and active(s), preferably DMSO/ethanol in amounts which are effective to dissolve the actives which are going to be administered the patient or subject. The actives are typically a PLA2 inhibitor and/or a metalloproteinase inhibitor and/or an antivenom (often all three actives), optionally in combination with one or more local anesthetics such as lidocaine, prilocaine and bupivacaine, among others), antibiotics and steroidal agents. The composition containing active(s) is then admixed with other components typically used to make these topical, buccal or sublingual formulations in the form of a salve, ointment, cream, liniment, tablet, films and/or gel. Topical formulations are often administered in combination with an occlusive dressing.

In the case of transdermal formulations, the base composition which comprises an absorption enhancement agent, often DMSO and an additional absorption enhancement agent, often ethanol, glycol and/or vegetable oil (most often ethanol), and active(s) are formulated as a transdermal patch, including a microneedle patch using methods which are well known in the art. Transdermal patches useful in the present invention include single-layer drug-in-adhesive patches, multi-layer drug in adhesive patches, reservoir patches, matrix patches and vapour patches, with a single-layer drug-in-adhesive patch being used most often. The single-layer drug-in-adhesive patch is often used, because the adhesive layer also contains the active. In this type of patch the adhesive layer not only serves to adhere the various layers together, along with the entire system to the skin, but is also responsible for the releasing of the active. The adhesive layer is surrounded by a temporary liner and a backing. This patch is typically often used with the present invention inasmuch as delivery can be made rapidly, providing immediate or near-immediate release. The backing of this patch may include a massage or pressure pump to provide lymphatic pressure and prevent or inhibit the spread of toxin at a wound or envenoming site. An occlusive dressing or bandage may also be used. A band may be included to hold the patch in place and maintain pressure to reduce the likelihood of spread of the toxin from the wound site.

The multi-layer drug-in-adhesive patch is similar to the single-layer system, but typically adds another layer of drug-in-adhesive, usually separated by a membrane (but not in all cases). One of the layers is for immediate release of the active and other layer is for controlled release of drug from the reservoir, which is advantageous to use over time, especially after an immediate bolus-dose is administered. This patch also has a temporary liner-layer and a permanent backing, which optionally includes a massage or pressure pump as described herein above. The active release from this depends on membrane permeability and diffusion of active molecules.

Reservoir patches may also be used in the present invention and these patches have a separate active layer. The active layer is typically a liquid compartment containing the active solution or suspension separated by the adhesive layer. The active(s) reservoir is totally encapsulated in a shallow compartment molded from a drug-impermeable metallic plastic laminate, with a rate-controlling membrane made of a polymer like vinyl acetate on one surface. This patch is also backed by the backing layer, which may include a massage or pressure pump for limiting the spread of toxin from the original site of the wound or insult. In this type of system the rate of release is zero order. A matrix patch, which also may be used, has an active layer of a semisolid matrix containing a active solution or suspension. The adhesive layer in this patch surrounds the active layer, partially overlaying it. Also known as a monolithic device. In the case of a vapour patch, the adhesive layer not only serves to adhere the various layers together but also to release vapour from an active for therapeutic effect. Vapour patches release essential oils for up to 6 hours and are mainly used for decongestion in combination with other therapy utilized herein.

Microneedle patches may also be used to administer compositions according to the present invention. Microneedle patches contain hundreds of tiny needles clustered on a miniature patch about the size of a contact lens. The microneedles are often made from biodissolvable sugar-like natural materials, and the composition including active to be delivered is mixed with this water-soluble material when fabricating the microneedles.

In preferred embodiments, the patch includes up to 2-3 cc of the composition which includes DMSO/alcohol and/or glycol and/or a vegetable oil or other absorption enhancement agent(s) and actives and includes a massage or pressure pump to reduce the spread of toxins from the site of the wound or insult or to put pressure on the reservoir containing active to enhance release of active. A band may also be included to put pressure on the patch to hold in place and/or to reduce the likelihood of spread of toxins from the wound site.

### EXAMPLES

DMSO+ethanol were combined at varying concentrations and refrigerated at 8° C. to minus 15° C. for prolonged periods with acceptable attributes at 10%, 20% and 25% ethanol (e.g. without freezing). The free-acid of varespladib (varespladib HCl) was dissolved at 100 mg/mL in DMSO, then diluted with ethanol to 75 mg/mL and 2 mL applied to an absorbent pad containing 4% lidocaine and applied to a simulated viper bite on the inventor's calf applied by penetration to 1 cm with 18 g hypodermic needles similar in depth to what would be experienced with a large viper's bite. There was no pain, discomfort or redness for the 24 hours the patch was applied to the wounded area. There was no visible residue of DMSO or varespladib on the skin at 24 hours.

### CONCLUSIONS

The DMSO-based compositions according to the present invention provide mixtures for rapid absorption, toxin neutralization and tissue stabilizing interventions.

The addition of a topical anesthetic reduces pain from the tissue insult and slows toxin spread.

Freezing point depression of DMSO insures rapid and effective transcutaneous absorption through direct contact, microneedle patch or other device-skin contact devices mitigating concerns for DMSO solidification in ambient conditions.

DMSO is particularly useful for its heretofore unrecognized, direct, anti-metalloprotease activity as well as its ability to carry large concentrations of drug substance through intact or compromised skin.

Because many snakebite envenoming injuries, for example, are intracutaneous, it is possible to use the topical formulations and device described herein to immediately treat local venom deposition while preventing and mitigating systemic envenoming syndromes. Further, because many snakebite envenomings result in vomiting that precludes effective use of oral medications, a drug-patch device such as this or a topical formulation could be lifesaving and could be further combined with a topical antiemetic.

The drug-patch device and topical compositions according to the present invention are uniquely useful for snakebite and arthropod envenoming injuries with local cytotoxic effects such as those caused by vipers (e.g. carpet vipers, rattlesnakes, lancehead vipers and copperheads) and elapids such as cobras. Long-term complications from these soft tissue injuries are mitigated by early, topical treatment.

### Further Examples

Topical formulations were tested on laboratory animals (mice) which had been administered snake venom (Oxyuranus scutellatus venom) at a concentration of venom 0.1 mg/kg subcutaneously (SC) to determine the impact of topical formulations on the therapeutic effect of envenomation. In a first control group of mice (3), all three mice died within 148 minutes after administration of venom. This is shown in Table 1, presented below. Note that all three test animals exhibited a rapid descent into weakness and paralysis prior to euthanasia.

TABLE 1
Controls 0.1 mg/kg Oxyuranus scutellatus venom SC

| | | Weakness | Cessation of | |
|---|---|---|---|---|
| | Weight | onset | breathing | |
| Subject | (grams) | (minutes) | (min) | Comments |
| 1 | 23 | 148 | 191 | Rapid descent into |
| | | | | weakness and paralysis |
| | | | | prior to euthanasia |
| 2 | 22 | 76 | 128 | Rapid descent into |
| | | | | weakness and paralysis |
| | | | | prior to euthanasia |
| 3 | 27 | 113 | 184 | Rapid descent into |
| | | | | weakness and paralysis |
| | | | | prior to euthanasia |
| Average | 24 | 112.3 | 167.7 | |
| Stdev | 2.65 | 36.0 | 34.53 | |

In a second group, three (3) mice were administered venom as per above, but after approximately ten(10) minutes (rapid treatment), the mice were topically administered the following formulations: Subject 4 (see Table 2, below): Varespladib 5 mg/ml in DMSO/EtOH (80:20 by volume) only; Subject 5: Varespladib 5 mg/ml in DMSO/EtOH w/simple sheen of 2.5%/2.5% lidocaine/prilocaine applied to skin before adding the Varespladib (<1% of total); Subject 6: Varespladib 5 mg/ml in DMSO/EtOH 5 mg/ml in Lido/prilocaine 2.5%/2.5% DMSO/EtOH 8%/2%.

TABLE 2
Rapid Treatment (~10 Minutes)

| | | Weakness | Cessation of | |
|---|---|---|---|---|
| | Weight | onset | breathing | |
| Subject | (grams) | (min) | (min) | Comments |
| 4 | 25 | N/A | >10080 | Never symptomatic/ |
| | | | | No signs |
| 5 | 21 | N/A | >10080 | Never symptomatic/ |
| | | | | No signs |
| 6 | 23 | N/A | >10080 | Never symptomatic/ |
| | | | | No signs |
| Average | 23.0 | 0.0 | >10080.0 | All survived to end |
| | | | | of study |
| Stdev | 2.00 | 0.00 | 0.00 | |

In a third group, two (2) mice were administered venom as per above, but the mice were rescued with substantial paralysis (one with severe hindlimb paralysis and the other with all four extremities and flaccid paralysis—near death at rescue) after approximately 2 hours (delayed rescue) by topical administration of the following formulations: Subject 7: Varespladib at a final concentration of 5.3% in lidocaine/prilocaine cream 2.5%/2.5% with 10% DMSO; Subject 8:Varespladib at a final concentration 5 mg/ml in lidocaine/prilocaine cream with DMSO/EtOH 8%/2%.

The results are presented in Table 3 herein below.

TABLE 3
Delayed Rescue Following Onset of Severe Systemic Signs

| | | Weakness | Cessation of | |
|---|---|---|---|---|
| | Weight | onset | breathing | |
| Subject | (grams) | (min) | (min) | Comments |
| 7 | 31 | 69 | >10080 | Total paralysis, then |
| | | | | walking within 8 hours |
| | | | | and slightly wobbly |
| | | | | for ~24 hours. |
| | | | | Completely normal BAR |
| | | | | by 72 hours |
| 8 | 26 | 117 | >10080 | Normal bright alert |
| | | | | responsive (BAR) by |
| | | | | 12 hours. |
| Average | 28.5 | 93.0 | 10080.0 | |
| Stdev | 3.54 | 33.94 | 0.00 | |

### Snake Venom Studies

Additional studies used historical controls from earlier studies with venom doses from same lots:

Daboia russelli: 1 mg/kg IP Varespladib 20%/Prinomastat 20% applied at 10 minutes and no signs symptoms manifested at >1440 min (n=1).

Crotalus scutulatus 1 mg/kg SC Varespladib 20%/Prinomastat 20% applied at 10 minutes and no signs symptoms manifested at >1440 min (n=1).

Micrurus fulvius 1 mg/kg SC Varespladib 20%/Prinomastat 20% applied at 10 minutes and no signs symptoms manifested at >1440 min (n=2).

Oxyuranus scutellatus severe neurotoxicity and 3-(5′-benzyl-2′-carbamoyl-[1,1′-biphenyl]-3-yl)-2-methylpropanoic acid as the racemic mixture in 80% DMSO/20% ethanol in <50% 2.5%/2.5% lidocaine/prilocaine cream+/−petrolatum cover (1 each). Four mice in study shows the following results:
#1 Cessation of breathing 64 minutes;
#2 Cessation of breathing 44 minutes;
#3 Cessation of breathing 104 minutes;
#4 Cessation of breathing 111 minutes.

The following conclusions can be drawn from the examples presented herein.

1. No topical cream/transdermal formulation has been made that can actually save a life after envenomation. The above experiments evidenced real life rescue of animals on death's doorstep.

2. The effect seems to be highly durable out of proportion to pill/IV. Both neurotoxic mice showed no signs of relapse for more than 96 hours.

3. Topical formulations superior to patch in ease of use and large surface area make this application as efficient as patch and substantially more cost effective.

4. The topical formulations of the present invention reversed systemic signs/symptoms of envenoming even when applied remotely to the bite site.

1. A composition for use in the treatment of a wound, injury, inflammatory condition or toxidrome comprising an absorption enhancement agent and at least one active selected from the group consisting of at least one PLA2 inhibitor and optionally, at least one antivenom, antibody, antibody fragment or antibody derivative and/or a metalloprotease inhibitor, wherein said composition is formulated for topical, transdermal, buccal or sublingual administration.

2. The composition according to claim 1 wherein said PLA2 inhibitor is varespladib, methyl varespladib (LY333013), AZD2716- 3-(5′-benzyl-2′-carbamoyl-[1,1′-biphenyl]-3-yl)-2-methylpropanoic acid as a racemic mixture or as the R or S enantiomer thereof, compound 4, LY433771 ((9-[(phenyl)methyl]-5-carbamoylcarbazol-4-yl) oxyacetic acid), a pharmaceutically acceptable salt thereof or a mixture thereof.

3. The composition according to either of claims 1-2 wherein said metalloprotease inhibitor is prinomastat, batimastat, marimastat, vorinostat or a mixture thereof.

4. The composition according to any one of claims 1-3 wherein said PLA2 inhibitor is varespladib.

5. The composition according to claim 4 comprising a metalloprotease inhibitor.

6. The composition according to any one of claims 1-5 comprising antivenom, antibody, antibody fragment or antibody derivative.

7. The composition according to any one of claims 1-6 comprising an antivenom.

8. The composition according to any one of claims 1-7 wherein said composition further includes at least one additional active selected from the group consisting of an antibiotic, an analgesic, a local anesthetic, a steroid or a mixture thereof.

9. The composition according to any one of claims 1-8 wherein the absorption enhancement agent is DMSO, an alcohol, glycol, vegetable fatty oil, a derivative thereof or a mixture thereof.

10. The composition according to any one of claims 1-8 wherein the absorption enhancement agent is DMSO, ethanol, propanol, isopropanol, butanol, isobutanol, 2-butanol, tertiary-butanol, ethylene glycol, diethylene glycol, diethylene glycol monoester or diester, glycol ethers (such as diethyleneglycol monomethyl ether, diethyleneglycol monoethylether/transcutol), triethylene glycol, PEG 200, PEG 400, PEG 600, PEG 800, PEG 1000, PEG 1500, PEG 2000, propylene glycol, dipropyleneglycol, tripropylene glycol, a propylene glycol monoester or diester, a polypropylene such as PPG 200, PPG 400, PPG 600, PPG 800, PPG 1000, PPG 1500, glycerin or an ester thereof (e.g., monoacetin, diacetin or triacetin, preferably triacetin), a vegetable oil such as castor oil, coconut oil, soybean oil, corn oil, olive oil, bees wax, or a mixture thereof.

11. The composition according to any one of claims 1-8 wherein the absorption enhancement agent is DMSO, ethanol, propylene glycol, polypropylene glycol (often PPG 200, 400 or 600), diethyleneglycol monoethylether, castor oil, coconut oil or a mixture thereof.

12. The composition according to any one of claims 1-8 wherein the absorption enhancement agent is DMSO, ethanol, stearic acid, sodium dodecyl sulfate (SDS), sodium lauryl sulfate (SLS), azone (I-dodecylazacycloheptan-2-one), carboxypolymethylene, polyoxyethylene fatty acid ester (polyolfatty acid ester), propylene glycol, a fatty acid, an ester of ricinoleic acid (e.g., methyl, ethyl, isopropyl, glycol, glyceryl, cetyl and octyldodecyl esters), castor seed oil, coconut oil, beeswax, shea butter, soybean oil, sweet almond oil, jojoba butter, tocopherol, citral, citonelloi, coumarin, farnesol, geraniol, limonene, linalool, isopropyl alcohol, mineral oil, polyoxyl 20 cetostearyl ether, propylene glycol, purified water, ammonia and mixtures thereof.

13. The composition according to any of claims 1-12 wherein said composition further includes a local anesthetic.

14. The composition according to claim 13 wherein said local anesthetic is lidocaine, prilocaine, bupivacaine or a mixture thereof.

15. The composition according to any one of claims 1-14 wherein said absorption enhancement agent comprises DMSO and ethanol.

16. The composition according to any of claims 1-15 wherein the absorption enhancement agent is a mixture of DMSO and ethanol in an amount ranging from 0.5% to 25% by weight of said composition.

17. The composition according to either of claims 15 or 16 wherein said DMSO and said ethanol are used in an amount effective to dissolve said actives while simultaneously depressing the freezing point of DMSO sufficiently to provide for immediate or near-immediate release for topical or transdermal administration of said active(s).

18. The composition according to any of claims 1-15 wherein said absorption enhancement agent is DMSO, ethanol and at least one additional absorption enhancement agent.

19. The composition according to any one of claims 1-18 wherein said absorption enhancement agent comprises DMSO and ethanol at a weight ratio ranging from 50:50 to 80:20.

20. The composition according to any of claims 1-19 which further comprises at least one additional active agent selected from the group consisting of a serine proteinase inhibitor, an acetylcholinesterase inhibitor (AChEI or AChEI combination), a spreading factor inhibitor, an NMDA receptor antagonist, an L-aminooxidase inhibitor, a muscarinic acetyl choline receptor (mAChR) inhibitor or a hyaluronidase inhibitor.

21. The composition according to any one of claims 1-20 comprising at least one local anesthetic selected from the group consisting of lidocaine, prilocaine and mixtures thereof.

22. The composition according to any of claims 1-21 which includes an effective amount of an antibiotic.

23. The composition according to any of claims 1-22 which includes an effective amount of a steroid.

24. The composition according to any of claims 1-23 which includes an effective amount of an analgesic and/or an antiemetic.

25. The composition according to any one of claims 1-24 comprising a metalloprotease inhibitor which is prinomastat.

26. The composition according to any one of claims 1-25 formulated as a salve, cream, ointment and/or gel for topical administration.

27. The composition according to claim 26 formulated as a topical cream.

28. The composition according to claim 26 or 27 used in combination with an occlusive dressing.

29. A composition comprising a PLA2 inhibitor selected from the group consisting of varespladib sodium, AZD2716 3-(5′-benzyl-2′-carbamoyl-[1,1′-biphenyl]-3-yl)-2-methylpropanoic acid as a racemic mixture or enantiomer thereof, compound 4 or a mixture thereof, optionally in combination with at least one metalloprotease inhibitor selected from the group consisting of prinomastat, marimistat and bastimistat wherein the PLA2 inhibitor and optional metalloprotease inhibitor are included in said composition at a molar ratio ranging from 1:1 to 1:5, often about 1:2, said composition further comprising DMSO or a combination of DMSO and ethanol (often 80:20), wherein the PLA2 inhibitor ranges from about 0.25 to 5% by weight of the composition, the optional metalloprotease inhibitor ranges from about 0.25 to 10% by weight of the composition with the remainder of the composition comprising DMSO alone or in combination with ethanol (often at 80:20 DMSO to ethanol).

30. The composition according to claim 29 including lidocaine, bupivacaine or prilocaine at a weight ratio ranging from 0.25% to 5%, often 0.5% to 4.5% or 1% to 3% of said composition to enhance analgesia and to slow lymphatic spread of toxins.

31. A composition comprising a PLA2 inhibitor (e.g., varespladib sodium, AZD2716 as a racemic mixture or enantiomer thereof, compound 4 or a mixture thereof as a PLA2 inhibitor, often varespladib Na+), optionally in combination with a metalloprotease inhibitor such as prinomastat, marimistat or bastimistat often at a molar ratio of PLA2 inhibitor to optional metalloprotease inhibitor ranging from 1:1 to 1:5, often about 1:2, wherein the PLA2 inhibitor ranges from about 0.25 to 5% by weight of the composition, the metalloprotease inhibitor ranges from about 0.25 to 10% by weight of the composition with the remainder of the composition comprising a thickening agent (carboxymethylene, hydrogenated castor oil, polysorbate 20/emulsifying wax 80%, carbomer) ranging from about 1% to about 20% by weight, about 60% to about 95% by weight of an emulsifier (polyoxyethylene oxide fatty acid ester, cetyl alcohol, hydrogenated castor oil), and optionally water, DMSO and/or DMSO/ethanol (80:20) as the remainder of the composition, wherein the pH of said composition ranges from abut 7.5 to 10.

32. The composition according to claim 31 including lidocaine, bupivacaine or prilocaine at a weight ratio ranging from 0.25% to 5%, often 0.5% to 4.5% or 1% to 3% to enhance analgesia and to slow lymphatic spread of toxins.

33. A method of treating an infection, wound, injury, inflammatory condition or toxidrome in a patient or subject in need comprising administering a composition according to any one of claims 1-32 to said patient or subject.

34. The method according to claim 33 wherein said composition is administered topically.

35. The method according to claim 30 wherein said composition is administered in combination with an occlusive dressing.

36. The method according to claim 29 wherein said composition is administered transdermally.

37. A method of treating an infection, wound, injury, inflammatory condition or toxidrome in a patient or subject in need comprising administering a composition comprising DMSO and ethanol and optionally at least one additional absorption enhancement agent in combination with at least one active selected from the group consisting of at least one PLA2 inhibitor, at least one metalloprotease inhibitor and an antivenom or an antibody, antibody fragment, antibody derivative or a mixture thereof, optionally in combination with an additional active selected from an antibiotic, an analgesic, local anesthetic and/or steroid, wherein the DMSO and ethanol and optional absorption enhancement agent are used in an amount effective to dissolve said actives while simultaneously depressing the freezing point of DMSO sufficiently to provide for immediate or near-immediate release for topical or transdermal administration of said active(s).

38. The method according to claim 37 wherein said composition is administered topically.

39. The method according to claim 38 wherein said composition is administered in combination with an occlusive dressing.

40. The method according to claim 37 wherein said composition is administered transdermally.
